(19) United States
(12) Patent Application Publication
(10) Pub. No.: US 2022/0039900 A1
(43) Pub. Date: Feb. 10, 2022

(54) USED DEVICE DETECTION

(71) Applicant: Canon U.S.A., Inc., Melville, NY (US)

(72) Inventor: Hitoshi Nakamura, Boston, MA (US)

(21) Appl. No.: 17/392,057

(22) Filed: Aug. 2, 2021

Related U.S. Application Data
(60) Provisional application No. 63/062,050, filed on Aug. 6, 2020.

(51) Int. Cl. A61B 90/00

(52) U.S. Cl. CPC A61B 90/08; A61B 2090/0804; A61B 2560/0285; A61B 2560/0214; A61B 2090/0814

(57) ABSTRACT

A single use medical guidance device capable of attachment to an patient, and designed to facilitate the precise placement of a medical guidance device upon and/or into the patient for inserting or otherwise attaching a medical tool upon the patient, wherein the medical guidance devise is capable or relaying the status of the device (used or new) to any third party user interface.

CROSS REFERENCE TO RELATED PATENT APPLICATIONS

This application claims priority from U.S. Provisional Patent Application No. 63/062050, filed on Aug. 6, 2020, in the United States Patent and Trademark Office, the disclosure of which is incorporated herein in its entirety by reference.

### FIELD OF THE DISCLOSURE

The present disclosure relates generally to apparatus and methods for medical application. More particularly, the subject disclosure is directed to apparatus configured to perform a biopsy or therapy support by puncture, using medical equipment, and more specifically, a detection system for determining if the equipment is new or previously used.

### BACKGROUND OF THE DISCLOSURE

Accurate and precise manipulation and orientation of medical tools is a critical factor in the successful use of these tools in surgical procedures. In particular, the accurate and precise orientation of a medical tool is especially critical in the insertion of a needle-like tool according to a plan based on medical images, such as computed tomography (CT) and Magnetic Resonance Imaging (MRI) in percutaneous interventions.

The general purpose of any needle guidance device is to direct the needle onto a path passing precisely through a selected insertion point to the target location within the patient's anatomy. This objective involves limiting the position and orientation to one azimuthal angle and one angle of elevation with respect to a selected insertion point upon the patient's skin surface. Selection of an insertion point, and both azimuthal and elevation angles, defines the needle trajectory into the patient's anatomy. The needle is able to slide axially along the chosen trajectory, to the desired depth.

In addition, the advent of one-time use instruments used in connection with the medical devices have spawned, and with it the need to identify if the instrument is new or has been previously used.

As it is vital to create instruments that are disposable (i.e. one-time use), while allowing the expensive underlying device to be reused, it is tantamount to be able to differentiate between a ‘used’ disposable instrument and an ‘unused’ disposable instrument, thus reducing the risk of contamination.

### SUMMARY

Thus, to address such exemplary needs in the industry, the presently disclosed device teaches a single use mountable medical guiding device comprising an adhesive pad having at least two sections of adhesive; at least two tabs each releasable attached to the adhesive pad wherein each of the at least two tabs are removable while the medical guiding device is being held in a mounting position on a subject; and a communication unit, wherein the medical guidance device is capable of communication with a user interface and further capable of reporting a status of the medical guidance device.

In another embodiment, the medical guidance device further comprises a sensor in the communication unit for determining the status of the medical guidance device.

In an additional embodiment of the medical guidance device, the status of the medical guidance device is either used or new.

The medical guidance device further contemplates comprising a memory in the communication unit for memorizing the status of the medical guidance device.

In another embodiment, the medical guidance device further comprises a battery in the communication unit for retaining the status of the medical guidance device. Furthermore, the battery maybe insulated by a removable insulator.

In yet another embodiment of the medical guidance device, a reset may be configured upon the communication unit for resetting the medical guidance device.

It is further contemplated that the medical guidance device communication unit is capable of communication with the user interface via wireless communication.

The subject application further teaches a method for using a single use mountable medical guiding device comprising: providing a mountable medical guidance device comprising: an adhesive pad having at least one sections of adhesive; at least one tab releasable attached to the adhesive pad wherein the at least two tab is removable while the medical guiding device is being held in a mounting position on a subject; and a communication unit, whereby the method includes: mounting the medical guidance device on the subject; connecting the medical guidance device to a user interface; and relaying a status of the medical guidance device to the user interface.

In addition, the method may further comprise a sensor in the communication unit for determining the status of the medical guidance device.

In another embodiment, the method directs the status of the medical guidance device to be either used or new.

In additional embodiments, the method further comprises a memory in the communication unit for memorizing the status of the medical guidance device.

It is further contemplated to include a battery in the communication unit for retaining the status of the medical guidance device, and that the battery may be insulated by a removable insulator.

In yet another embodiment, the method further comprises incorporating a reset configured upon the communication unit for resetting the medical guidance device.

These and other objects, features, and advantages of the present disclosure will become apparent upon reading the following detailed description of exemplary embodiments of the present disclosure, when taken in conjunction with the appended drawing guidance device 10, and provided paragraphs.

### BRIEF DESCRIPTION OF THE DRAWIGUIDANCE DEVICE 10

Further objects, features and advantages of the present invention will become apparent from the following detailed description when taken in conjunction with the accompanying figures showing illustrative embodiments of the present invention.

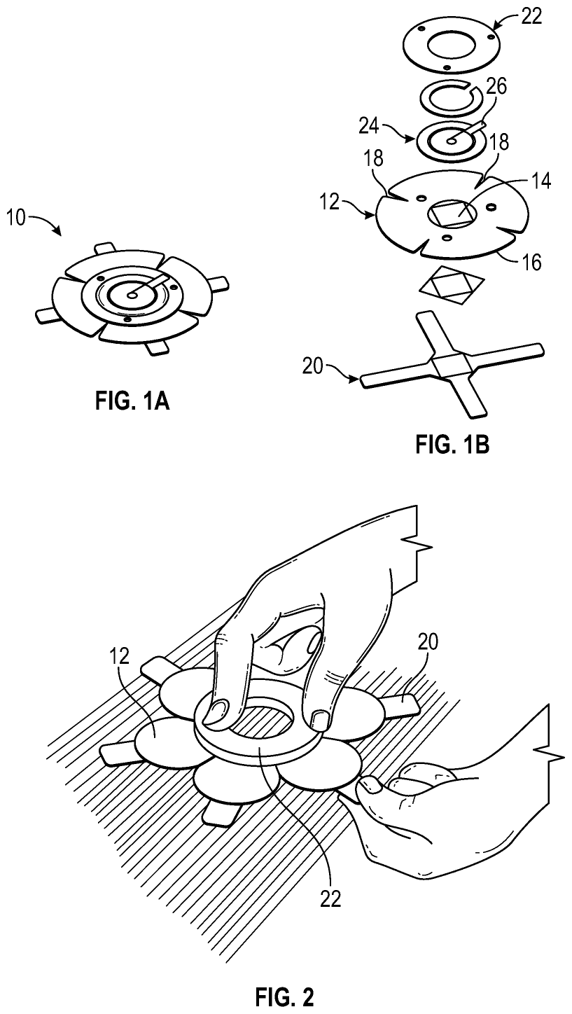

FIG. 1a is is a perspective view of an exemplary medical guidance device in accordance with one or more embodiment of the subject innovation.

FIG. 1b depicts an exploded perspective view of an exemplary medical guidance device in accordance with one or more embodiment of the subject innovation.

FIG. 2 provides a perspective view of an exemplary medical guidance device applied to a subject, in accordance with one or more embodiment of the subject innovation.

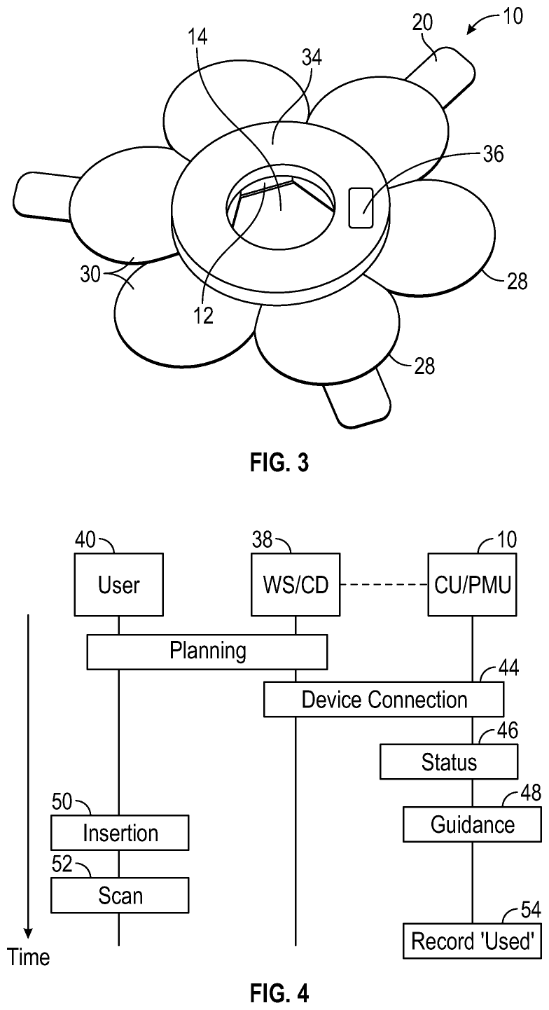

FIG. 3 provides is a top view of an exemplary medical guidance device in accordance with one or more embodiment of the subject innovation.

FIG. 4 a flow diagram depicting use of an exemplary medical guidance device in accordance with one or more embodiment of the subject innovation.

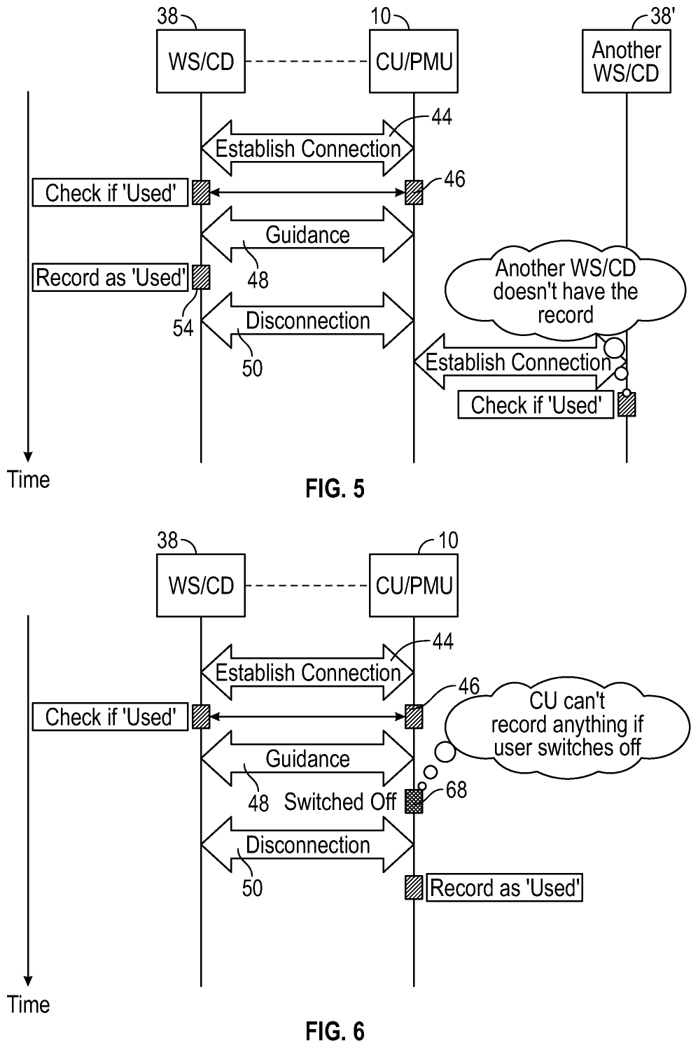

FIG. 5 a flow diagram depicting use of an exemplary medical guidance device in accordance with one or more embodiment of the subject innovation.

FIG. 6 a flow diagram depicting use of an exemplary medical guidance device in accordance with one or more embodiment of the subject innovation.

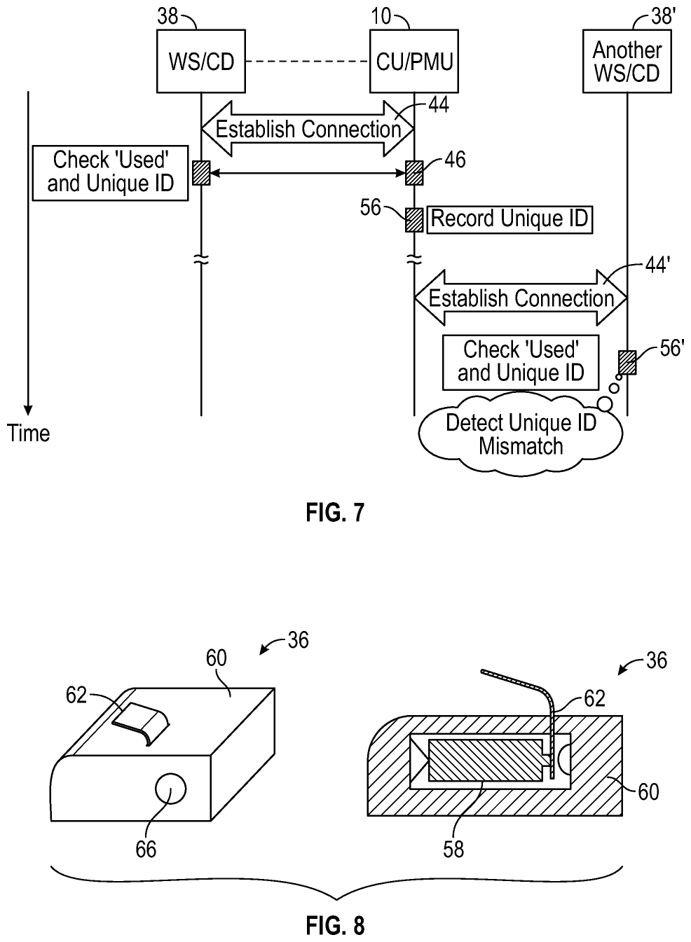

FIG. 7 a flow diagram depicting use of an exemplary medical guidance device in accordance with one or more embodiment of the subject innovation.

FIG. 8 provides a side view of a communication unit incorporated in an exemplary medical guidance device in accordance with one or more embodiment of the subject innovation.

Throughout the FIGS., the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the FIGS., it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings guidance device 10 herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

The present disclosure details a one-time use medical guidance device 10 capable of attachment to an patient, and designed to facilitate the precise placement of a medical guidance device upon and/or into the patient for inserting or otherwise attaching a medical tool upon the patient. FIG. 1a is provides a perspective view of the medical guidance device 10, with FIG. 1b illustrating an exploded view of the same guidance device 10 in FIG. 1a. The medical guidance device 10 incorporates an adhesive pad 12 with an opening 14. The opening 14 may be in the center of the adhesive pad 12 or in any known location required by the guidance device 10 being placed. In one embodiment, the opening 14 is an oval or circular opening substantially in the center of the medical guidance device 10 and of a size sufficient for a user to access a target position on a patient for a biopsy, ablation, and/or other procedures.

The adhesive pad 12 comprises of adhesive 16 on the surface of the adhesive pad 12 coming in contact with the patent. The adhesive 16 may be found on a portion of the adhesive pad 12, or encompass the entire surface of the adhesive pad 12. In the subject embodiment illustrated in FIG. 1b, the adhesive 16 is located on a portion of the adhesive pad 12, in the shape of a cross. The adhesive pad 12 may be perforated and/or slotted 18 to create two or more sections. FIG. 1b shows an embodiment with four sections for reference. A number of tabs 20 equals and covers the adhesive 16 and extends releasably past the edge of the adhesive 16 so they are accessible by the user when the guidance device 10 is placed upon the patient, as can been seen FIG. 2. The tabs 20 may also fold back upon themselves. The tabs 20 are formed of a material that is able to bend backwards such that it can be pulled out between a patient's skin and the base of the guidance device 10, while the guidance device 10 is positioned upon the patient.

The adhesive pad 12 is oriented such that the tab 20 and adhesive 16 are facing downward with respect to the guide 22 being in contact with the adhesive pad 12, located on the opposing side. Between the guide 22 and the adhesive pad 12 is a transparent target 24 with an opening in the location of alignment onto the patient. The transparent target 24 is perforated along the edge of the adhesive pad 12 opening 14 with a pull tab 26. Pulling the pull tab 26 of the transparent target 24, will result in removal of the transparent target 24, allowing greater access to the patient.

In one embodiment of the subject guidance device 10, shown in FIG. 3, the adhesive pad 12 is split into fully separate lobes 28. The adhesive 16 may be present in the fanned area 30 and not extend into the arm 32. The lobes 28 may vary in size and shape to accommodate the desired mounting location upon the patient's skin. Two or more lobes 28 may be aligned around the opening 14. An example utilizing 4 lobes is shown in FIG. 3, with the opening 14.

In another embodiment, only a subset of the lobes 28 may be revealed for the initial placement. This subset will be sufficient to adhere the guidance device 10 to the patient. The tabs 20 on the remaining lobes 28 may remain intact. The device may be used and removed from the patient's skin, multiple times using the other lobes 28. The tabs 20 on the remaining lobes 28 may be removed, exposing fresh adhesive 16. Accordingly, a second or multiple placement(s) of the device 10 can be achieved.

In yet another embodiment seen in FIG. 3, a base including a rigid ring 34 adhered to the upper portion of the adhesive pad 12 opposite the patient contacting face. The rigid ring 34 will allow the user to apply light pressure to the adhesive pad 12, holding it in place during removal of the tabs 20, without deforming or displacing the guidance device 10. The rigid ring 34, may be adhered to the adhesive pad 12 through adhesive, hardware such as screws, or a process such as heat sealing. The rigid ring 34 may act as a base of the medical guidance device 10 and may be of any shape but preferably does not block the opening 14. The rigid ring 34 may consist of the rigid component where the guidance portion of the medical guidance device 10 is attached directly to, and, in some cases, is permanently bonded to the rigid ring 34. Alternatively, the rigid ring 34 is configured to connect the guidance portion of the medical guidance device 10 after the adhesive pad 12 is in place on a patient. In some examples, there are mating features that are used to connect the rigid ring 34 to the guidance device 10, including, for examples, snaps, adhesives, threads, keyways, etc.

Furthermore, the guidance device 10 incorporates a communication unit 36 capable of communicating with a user interface 38. In one embodiment, the communication unit 36 is a sensor 60, however, a plethora of other communication units may be incorporated, including a dongle, etc. In addition, the communication unit 36 may include memory 42. The communication may be wireless (e.g. Bluetooth, wi-fi) or via wire or other information relaying means. The communication may be one-way or two-way, between the communication unit 36 and user interface 38.

The guidance device 10 is connected wirelessly to the user interface 38, though it is contemplated that they may be wired communication as well. As provided in FIG. 4, communication between the guidance device 10 and interface 38 comprises of four steps: establishment of connection between the two 44; reading the status of the guidance device 10 (‘used’ or ‘new’) 46; guidance control 48 through the guidance device 10; and disconnection 50.

In general, establishment of connection step 44 and disconnection step 50 are supported by communication protocols (e.g. Bluetooth). In reading the status 46 of the guidance device 10, the user interface 38 reads the status from the guidance device 10. If the guidance device 10 doesn't have a ‘Used’ status, the user interface 38 identifies the guidance device 10 as not ‘used’ and is ready for use. If the guidance device 10 has a ‘used’ status, the user interface 38 notifies user 40 of the status.

Since wireless communication doesn't connect physically, an unexpected (third party) guidance device could be connected to the user interface 38. This notification makes the user 40 confirm the correct guidance device 10 is connected. When the guidance device 10 shows a ‘used’ status, user 40 may have an option to remove the status and continue with the procedure. This is because software bugs could detect wrong/unexpected ‘used’ status, allowing for greater flexibility to the end user 40.

Before the guidance 48 step begins, the guidance device 10 accepts user 40 input to define an insertion plan 50. In the guidance 48 step, the user interface 38 calculates insertion angle and depth of needle insertion based on the user 40 defined plan, and transfers the plans to the guidance device 10. The guidance device 10 guides the insertion angle with LEDs on it. The user 40 inserts the needle along the guidance guidelines and scans images 52 (e.g. CT) to achieve a visual results. When the user 40 confirms that the needle(s) are inserted accurately, the guidance 48 is completed, and the guidance device 10 receives 54 the ‘used’ status. Therefore, the guidance device 10 records ‘used’ after the guidance 48 is completed. This record is stored in the non-volatile memory 42 in the guidance device 10. Which may be kept with or without a power supply.

By way of example, FIG. 5 shows an incomplete solution to the subject problem, further illustrating the need for the above taught complete solution. Here the user interface 38 memorizes the ‘used’ status in place of the guidance device 10. The shortfall here is revealed when a third party user interface 38′ (e.g.—another hospital or facility) is attached to the guidance device 10, which cannot know the ‘used’ status unless the information is shared between the user interfaces.

FIG. 6 depicts yet another incomplete solution. Here the guidance device 10 is branded ‘used’ after disconnection. However, this solution doesn't work when the device is turned off 68 without memorization. If the device has a power switch, a user may recycle the medical device 10, which could reset the status.

In an additional embodiment, the workflow in FIG. 7 depict how the subject innovation may communicate with a third party user interface 38′, wherein the communication unit 36 memorizes unique ID 56 (e.g. Bluetooth MAC Address) of medical device 10 when connected. When a medical device 10 connects to the user interface 38′ it checks the status and unique ID 56′ of the medical device 10. If the memorized unique ID 56′ doesn't match connecting user interface's one, the user interface 38 notifies user 40 to confirm the connected medical device 10. This solution enables the suppression of redundant notifications because reconnection the same user interface 38 doesn't cause notifications. Wireless communication could be interrupted. Additionally, the user interface 38 software could crash. If a device records ‘used’ status before guidance step, it conflicts to real status.

In order to facilitate memory 42 in the medical device 10, the medical device is fitted with a battery 58 to supply energy to the sensor 6o for transmitting the status of the medical device 10. In order to preserve battery life, a tab 62 may be inserted as an electrical insulator between a pole of the battery 58 and the battery connect 64. When an end user pulls off tab 62, the battery 58 supplies power to the medical device 10 and firmware runs on user interface 38. The medical device 10 continues to operate until battery 58 loses power. The guidance device 10 and/or communication unit 36 may also have a reset button 66 to establish or reestablish connection with user interface 38. Firmware broadcasts advertising signal for a while after the reset button 66 is pressed. The user interface 38 may detect the firmware broadcast and establish a connection. This process saves battery life because broadcasting spends power, thus killing the battery. The medical device 10 transmits values detected by sensor 6o through the communication unit 36 to the user interface 38.

Thus, in use, there is provided a method for guiding a medical instrument using the medical guidance device 10 as described herein. In a surgical workflow, a user will often mark a target location on a patient where the medical instrument should go, where this target location is based on, for example, prior imaging information about the position of interest within the patient (e.g., a tumor to be biopsied or ablated). The user holds the medical guidance device 10 at a target location on a patient, where this may be simplified by the presence of a transparent target 24 indicating the exact position on the medical guidance device 10 that should correspond with the target location. While the medical guidance device 10 is held at the target location, the user mounts the medical guidance device 10 using the adhesive 16. To do this, the user simply pulls the tabs 20 from the underside of the adhesive pads 12 while maintaining the position of the medial guidance device 10 on the patient. Dependent on whether there will be a need to reposition the device as well as how firmly the user wants to mount the medical guidance device 10, all or a subset of the tabs 20 will be pulled to adhere the medical guidance device 10 onto the patient. After the medical guidance device 10 is mounted, the user can guide a medical instrument to a position from the target location.

The present application hereby incorporates by reference, in their entirety, U.S. Pat. No. 9,222,996 titled “Needle Placement Manipulator with Two Rotary Guides”, and United States Patent Publication No. 2018/0228568 titled “Medical Guidance Apparatus”. In particular, the medical guidance device as described herein may include the described needle placement manipulator or the medical guidance apparatus as the guidance portion and base and thus may be fixed on a patient using the tabs and release liner as described herein.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A single use mountable medical guiding device comprising:
an adhesive pad having at least two sections of adhesive;
at least two tabs each releasable attached to the adhesive pad wherein each of the at least two tabs are removable while the medical guiding device is being held in a mounting position on a subject; and
a communication unit,
wherein the medical guidance device is capable of communication with a user interface and further capable of reporting a status of the medical guidance device.

2. The medical guidance device of claim 1, further comprising a sensor in the communication unit for determining the status of the medical guidance device.

3. The medical guidance device of claim 1, wherein the status of the medical guidance device is either used or new.

4. The medical guidance device of claim 1, further comprising a memory in the communication unit for memorizing the status of the medical guidance device.

5. The medical guidance device of claim 1, further comprising a battery in the communication unit for retaining the status of the medical guidance device.

6. The medical guidance device of claim 6, wherein the battery is insulated by a removable insulator.

7. The medical guidance device of claim 1, further comprising a reset configured upon the communication unit for resetting the medical guidance device.

8. The medical guidance device of claim 1, wherein the communication unit communicated with the user interface via wireless communication.

9. A method for using a single use mountable medical guiding device comprising:
providing a mountable medical guidance device comprising:
an adhesive pad having at least one sections of adhesive;
at least one tab releasable attached to the adhesive pad wherein the at least two tab is removable while the medical guiding device is being held in a mounting position on a subject; and
a communication unit,
mounting the medical guidance device on the subject;
connecting the medical guidance device to a user interface; and
relaying a status of the medical guidance device to the user interface.

10. The method of claim 9, further comprising a sensor in the communication unit for determining the status of the medical guidance device.

11. The method of claim 9, wherein the status of the medical guidance device is either used or new.

12. The method of claim 9, further comprising a memory in the communication unit for memorizing the status of the medical guidance device.

13. The method of claim 9, further comprising a battery in the communication unit for retaining the status of the medical guidance device.

14. The method of claim 13, wherein the battery is insulated by a removable insulator.

15. The method of claim 9, further comprising a reset configured upon the communication unit for resetting the medical guidance device.

16. The method of claim 9, wherein the communication unit communicated with the user interface via wireless communication.
